(19) United States
(12) Patent Application Publication
(10) Pub. No.: US 2025/0108188 A1
(43) Pub. Date: Apr. 3, 2025

(54) EXPANDABLE VASCULAR INTROCUCER

(71) Applicant: Oscor Inc., Palm Harbor, FL (US)

(72) Inventors: Andrew J. Enerson, Hudson, FL (US); Jason Schinis, St Petersburg, FL (US); Kameron Ely, Palm Harbor, FL (US)

(21) Appl. No.: 18/897,129

(22) Filed: Sep. 26, 2024

Related U.S. Application Data
(60) Provisional application No. 63/541,310, filed on Sep. 29, 2023.

(51) Int. Cl. A61M 25/00

(52) U.S. Cl. CPC A61M 25/0023

(57) ABSTRACT

In various examples, a vascular introducer is described. The vascular introducer includes a proximal housing defining an axial passageway. An elongated tubular sheath extends distally from the proximal housing and includes a sheath wall defining a central lumen. The central lumen of the tubular sheath includes an unexpanded configuration and an expanded configuration. The central lumen in the unexpanded configuration includes a first inner diameter, and the central lumen in the expanded configuration includes a second inner diameter, the second inner diameter being larger than the first inner diameter. The tubular sheath includes a plurality of circumferentially spaced apart elongated struts disposed partially within the sheath wall, such that an exposed portion of each of the plurality of struts extends radially inwardly from an inner surface of the sheath wall into the central lumen.

### CROSS REFERENCE TO RELATED APPLICATIONS

This application claims the benefit of priority to U.S. Provisional Application Ser. No. 63/541,310, filed on Sep. 29, 2023, entitled “EXPANDABLE VASCULAR INTRODUCER,” each of which is incorporated by reference herein in its entirety.

### BACKGROUND

The subject invention is directed to surgical instrumentation, and more particularly, to a vascular introducer that includes a dynamically expandable sheath which expands and contracts in response to the introduction of differently sized dilators.

Minimally invasive, percutaneous surgical access has become the standard of care for many vascular surgeries. Vascular access devices for minimally invasive percutaneous interventional procedures, including endovascular procedures, are known in the art. To reduce trauma, access devices having a relatively small sheath size are typically employed during a percutaneous procedure.

However, some percutaneous interventional procedures involve the use of large devices that require relatively large sheaths to deliver them to the intended site in the body. Sheath sizes for such procedures can vary from about 12 F to about 34 F (about 0.158 inches to about 0.445 inches in diameter). As a result, multiple intermediate sheath sizes must be successively introduced and removed in order to reach the largest introducer size required. This can lead to increased trauma at the access site, such as vessel damage, increased bleeding, and increased risk of infection. Studies have shown that the ability to gain initial access through a smaller outer diameter sheath that can later expand to a larger outer diameter to allow passage of larger devices and therapeutic delivery systems can be extremely beneficial to both allow for placement of devices through difficult anatomy and to reduce risk of complications from the procedure to the patient.

To address these issues, access devices have been developed with sheaths that can be expanded within the body so that their removal from the operative site is not required to upsize. Still, there is a need for more advanced and improved expandable sheaths. The subject invention satisfies that need.

### OVERVIEW

This overview is intended to provide an overview of subject matter of the present patent document. It is not intended to provide an exclusive or exhaustive explanation of the invention. The detailed description is included to provide further information about the present patent document.

The present inventors have recognized, among other things, that the present subject matter can be used to provide a vascular introducer with the ability to expand and contract in response to the insertion of different diameter therapeutic devices, thereby avoiding the need to remove the initial access introducer from the surgical access point in order to introduce larger sheaths/introducers thereafter. In various examples, the present inventive subject matter is advantageous in that it provides a vascular introducer including a lubricious material in the device lumen or inner diameter (ID) and/or various rib shapes and configurations to allow for easier passage of dilators and devices, for instance, due to decreased frictional resistance and insertion force during passage of a device within the vascular introducer. In some examples, the present inventive subject matter provides for a vascular introducer including one or more various rib shape designs to allow for improved strength and performance within a limited size/wall thickness. To better illustrate the devices described herein, a non-limiting list of examples is provided here:

Example 1 can include subject matter that can include a vascular introducer including a proximal housing defining an axial passageway. An elongated tubular sheath extends distally from the proximal housing and includes a sheath wall defining a central lumen. The central lumen of the tubular sheath includes an unexpanded configuration and an expanded configuration. The central lumen of the tubular sheath in the unexpanded configuration includes a first inner diameter and the central lumen of the tubular sheath in the expanded configuration includes a second inner diameter, the second inner diameter being larger than the first inner diameter. The tubular sheath includes a plurality of circumferentially spaced apart elongated struts disposed partially within the sheath wall, such that an exposed portion of each of the plurality of struts extends radially inwardly from an inner surface of the sheath wall into the central lumen.

In Example 2, the subject matter of Example 1 is optionally configured such that a cross section of at least one of the plurality of struts includes a substantially rectangular shape.

In Example 3, the subject matter of Example 1 or 2 is optionally configured such that a cross section of at least one of the plurality of struts includes a substantially triangular shape.

In Example 4, the subject matter of any one of Examples 1-3 is optionally configured such that a cross section of at least one of the plurality of struts is substantially star-shaped.

In Example 5, the subject matter of any one of Examples 1-4 is optionally configured such that a cross section of at least one of the plurality of struts is substantially U-shaped.

In Example 6, the subject matter of any one of Examples 1-5 is optionally configured such that a cross section of at least one of the plurality of struts is substantially I-shaped.

In Example 7, the subject matter of any one of Examples 1-6 is optionally configured such that the central lumen of the tubular sheath is configured to transition between the first inner diameter and the second inner diameter in response to introduction of a device within the central lumen. The device includes an outer diameter that is greater than the first inner diameter of the central lumen of the tubular sheath.

In Example 8, the subject matter of any one of Examples 1-7 optionally includes a body portion extending distally from the proximal housing and defining an axial bore in communication with the axial passageway of the proximal housing, wherein the tubular sheath extends distally from the axial bore of the body portion.

In Example 9, the subject matter of any one of Examples 1-8 is optionally configured such that the exposed portion of each of the plurality of struts provides a contact area along which a device inserted within the central lumen slides. The contact area is less than a surface area of an interior of the tubular sheath to decrease frictional resistance experienced by the device inserted within the central lumen.

In Example 10, the subject matter of Example 9 is optionally configured such that at least the contact area of each of the plurality of struts is formed from a lubricious material to decrease frictional resistance experienced by the device inserted within the central lumen.

In Example 11, the subject matter of any one of Examples 1-10 is optionally configured such that the body portion is formed from a flexible material.

In Example 12, the subject matter of any one of Examples 1-11 is optionally configured such that the sheath wall is formed from an expandable polymeric material.

Example 13 can include, or can optionally be combined with any one of Examples 1-12 to include subject matter that can include a vascular introducer. A proximal housing defines an axial passageway. An elongated tubular sheath extends distally from the proximal housing and includes a sheath wall defining a central lumen. The sheath wall is formed from an expandable polymeric material. The central lumen of the tubular sheath includes an unexpanded configuration and an expanded configuration. The central lumen of the tubular sheath in the unexpanded configuration includes a first inner diameter, and the central lumen of the tubular sheath in the expanded configuration includes a second inner diameter. The second inner diameter is larger than the first inner diameter. The tubular sheath includes a plurality of circumferentially spaced apart elongated struts disposed partially within the sheath wall, such that an exposed portion of each of the plurality of struts extends radially inwardly from an inner surface of the sheath wall into the central lumen. The exposed portion of each of the plurality of struts provides a contact area along which a device inserted within the central lumen slides. The contact area is less than a surface area of an interior of the tubular sheath. At least the contact area of each of the plurality of struts is formed from a lubricious material, such that frictional resistance experienced by the device inserted within the central lumen is decreased.

In Example 14, the subject matter of Example 13 is optionally configured such that a cross section of at least one of the plurality of struts includes a substantially rectangular shape.

In Example 15, the subject matter of Example 13 or 14 is optionally configured such that a cross section of at least one of the plurality of struts includes a substantially triangular shape.

In Example 16, the subject matter of any one of Examples 13-15 is optionally configured such that a cross section of at least one of the plurality of struts is substantially star-shaped.

In Example 17, the subject matter of any one of Examples 13-16 is optionally configured such that a cross section of at least one of the plurality of struts is substantially U-shaped.

In Example 18, the subject matter of any one of Examples 13-17 is optionally configured such that a cross section of at least one of the plurality of struts is substantially I-shaped.

In Example 19, the subject matter of any one of Examples 13-18 is optionally configured such that the central lumen of the tubular sheath is configured to transition between the first inner diameter and the second inner diameter in response to introduction of a device within the central lumen. The device includes an outer diameter that is greater than the first inner diameter of the central lumen of the tubular sheath.

Example 20 can include, or can optionally be combined with any one of Examples 1-19 to include subject matter that can include a vascular introducer. A proximal housing defines an axial passageway. A body portion extends distally from the proximal housing and defines an axial bore in communication with the axial passageway of the proximal housing. The body portion is formed from a flexible material. An elongated tubular sheath extends distally from the axial bore of the body portion and includes a sheath wall defining a central lumen. The sheath wall is formed from an expandable polymeric material. The central lumen of the tubular sheath includes an unexpanded configuration and an expanded configuration. The central lumen of the tubular sheath in the unexpanded configuration includes a first inner diameter, and the central lumen of the tubular sheath in the expanded configuration includes a second inner diameter. The second inner diameter is larger than the first inner diameter. The tubular sheath includes a plurality of circumferentially spaced apart elongated struts disposed partially within the sheath wall, such that an exposed portion of each of the plurality of struts extends radially inwardly from an inner surface of the sheath wall into the central lumen. The exposed portion of each of the plurality of struts provides a contact area along which a device inserted within the central lumen slides. The contact area is less than a surface area of an interior of the tubular sheath. At least the contact area of each of the plurality of struts is formed from a lubricious material, such that frictional resistance experienced by the device inserted within the central lumen is decreased.

### BRIEF DESCRIPTION OF THE DRAWINGS

FIG. 1 is a perspective view of a vascular introducer constructed in accordance with at least one example of the invention, wherein a portion of a tubular sheath is shown in cross-section to reveal a central lumen thereof.

FIG. 2 is a side elevational view of the vascular introducer of FIG. 1, with a dilator partially inserted therein, and wherein a portion of the tubular sheath is shown in cross-section to illustrate how the tubular sheath is dynamically expanded by the shaft of the dilator from a first inner diameter to a second inner diameter.

FIG. 3 is an enlarged localized view taken from FIG. 2.

FIG. 4 is a side elevational view of a proximal housing of the vascular introducer of FIG. 1.

FIG. 5 is a perspective view of the proximal housing of the vascular introducer of FIG. 1.

FIG. 6 is a side elevational view of a body portion of the vascular introducer of FIG. 1.

FIG. 7 is a perspective view of the body portion of the vascular introducer of FIG. 1.

FIG. 8 is an enlarged localized view of taken from FIG. 2, illustrating the connection between a proximal flange of proximal housing and an axial bore of the body portion.

FIG. 9 is a cross-sectional view of an expandable tubular sheath in accordance with at least one example of the invention.

FIG. 10 is a cross-sectional view of the tubular sheath of FIG. 9 in a fully expanded configuration.

FIG. 11 is a cross-sectional view of an expandable tubular sheath in accordance with at least one example of the invention.

FIG. 12 is a cross-sectional view of an expandable tubular sheath in accordance with at least one example of the invention.

FIG. 13 is a cross-sectional view of an expandable tubular sheath in accordance with at least one example of the invention.

FIG. 14 is a cross-sectional view of an expandable tubular sheath in accordance with at least one example of the invention.

FIG. 15 is a cross-sectional view of an expandable tubular sheath in accordance with at least one example of the invention.

### DETAILED DESCRIPTION

The present invention relates generally to providing a vascular introducer for introduction of dilators and other devices within a patient. More specifically, the present invention relates to a vascular introducer that can expand and contract inner and outer diameters in response to the introduction of differently-sized dilators and other devices. As such, it is contemplated herein that the present inventive subject matter can be used to provide a vascular introducer that is minimally invasive during insertion within a patient but can then expand to larger diameters, if desired, to allow for insertion of a larger dilator or other medical device within the vascular introducer. In some examples, such a device can include a medical device; however, the present invention is not intended to be so limited as devices other than medical devices are contemplated herein.

In some examples, a vascular introducer is provided with the ability to expand and contract in response to the insertion of different diameter therapeutic devices, thereby avoiding the need to remove the initial access introducer from the surgical access point in order to introduce larger sheaths/introducers thereafter.

In some examples, a lubricious material is provided within the device lumen or inner diameter (ID) of the vascular introducer to allow for easier passage of dilators and other devices. In some examples, the vascular introducer includes an expandable material to allow for expansion of the lumen or ID, and such an expandable material tends to be sticky. Conventional expandable sheaths have the harder ribs flush with or disposed completely within the stretchy expandable material. However, in some examples, the vascular introducer of the present inventive subject matter includes one or more ribs protruding inwardly from a sheath wall (formed from the expandable material) into the introducer lumen to separate a device being inserted within the lumen of the vascular introducer from the expandable material, thereby limiting, if not eliminating, contact between the device being inserted and the potentially sticky expandable material of the sheath wall.

In some examples, the one or more ribs can be formed from a lubricious material (which, in at least some examples, is more lubricious than the expandable material) to allow the device to slide past the one or more ribs and through the lumen of the vascular introducer relatively easily. In some examples, the expandable sheath of the present inventive subject matter provides a separation between the introduced devices and the potentially stickier expandable material of the sheath wall, which can reduce insertion force and friction during passage. In various examples, the present inventive subject matter includes various rib shape designs to allow for improved strength and performance within a limited size/wall thickness.

In some examples, the construction and/or configuration of the one or more ribs within the sheath wall allows for more expandable material to be used within the sheath wall, which allows for more expansion with less force. In some examples, the present inventive subject matter provides for a vascular introducer with simpler construction, thereby allowing for easier manufacturing of the present vascular introducer as compared to conventional introducers.

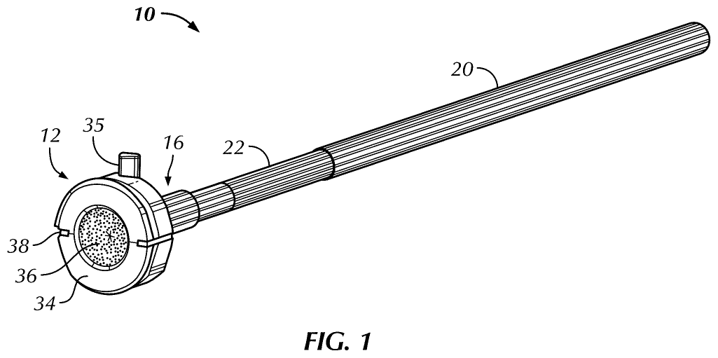

Referring now to the drawings wherein like reference numerals identify similar structural elements or features of the subject invention, there is illustrated in FIG. 1 a new and useful vascular introducer constructed in accordance with a preferred embodiment of the subject invention and designated generally by reference numeral 10. In brief, the vascular introducer 10 is designed to be expanded within the body so that its removal from the operative site is not required to accommodate the placement of a larger sheath to introduce a larger sized instrument or device during a percutaneous interventional procedure.

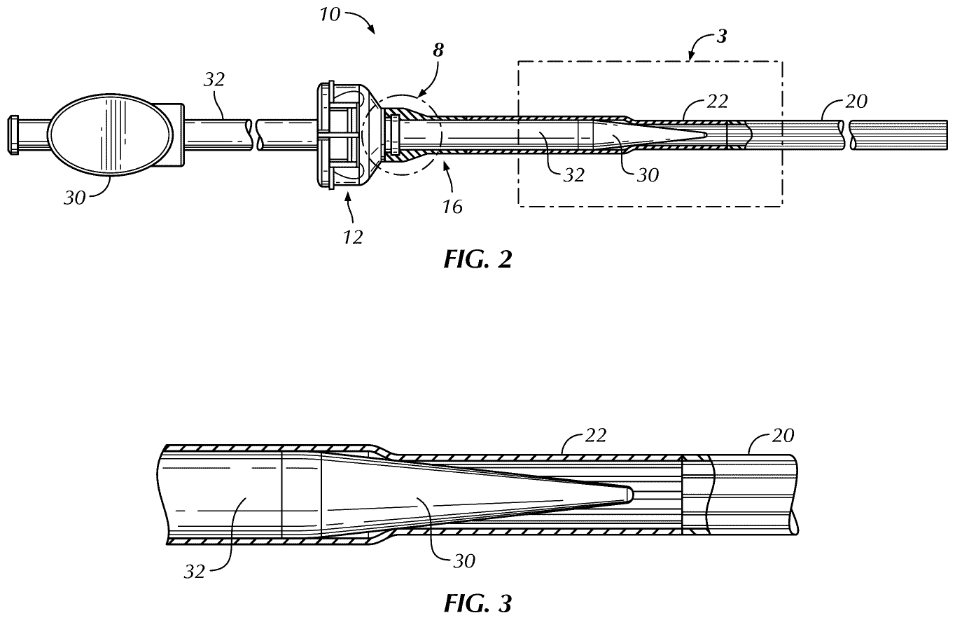

The vascular introducer 10 includes a proximal housing 12 defining an axial passageway 14 (see FIGS. 5 and 8) and a body portion 16 extending distally from the proximal housing 12 and defining an axial bore 18 (see FIGS. 6 and 7) in communication with the axial passageway of the proximal housing, as best seen in FIG. 8. The vascular introducer 10 further includes an elongated tubular sheath 20 that extends distally from the axial bore 18 of the body portion 16 and defines a central lumen 22, as best seen in FIG. 2.

As explained in more detail below and as illustrated in FIGS. 2 and 3, the axial bore 18 of the body portion 16 and the central lumen 22 of the tubular sheath 20 are adapted and configured to transition or otherwise expand between a first inner diameter ID₁ and a second inner diameter ID₂ in response to the introduction of a dilator 30 having a shaft 32 with an outer diameter OD that is greater than the first inner diameter ID₁ of the axial bore 18 of the body portion 16 and the central lumen 22 of the tubular sheath 20. The dilator 30 can include a central bore for accommodating a guidewire or a stylet, and the dilator shaft 32 has a tapered distal tip for facilitating its percutaneous insertion.

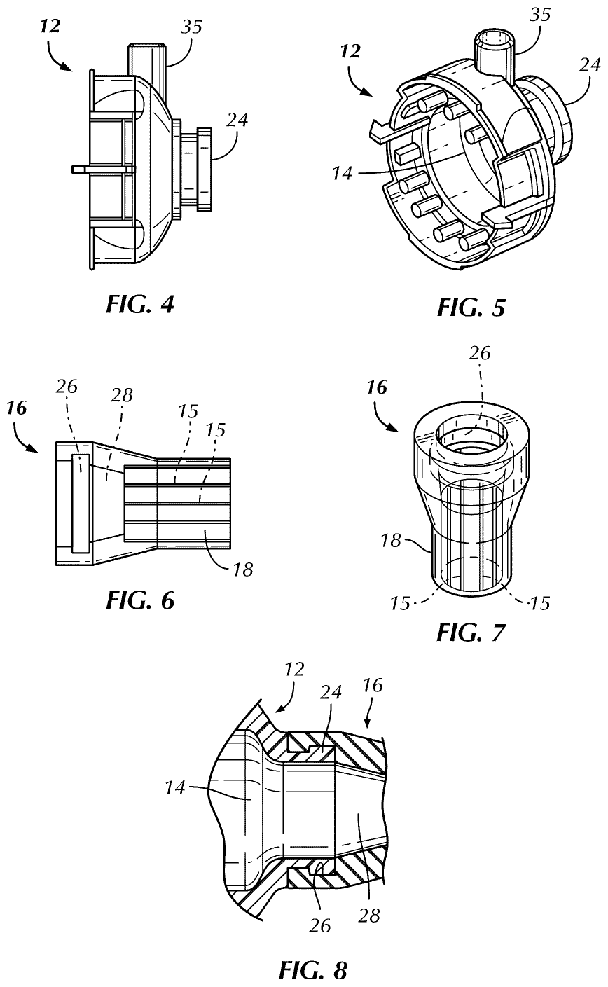

Referring to FIGS. 4 through 8, the proximal housing portion 12 of vascular introducer 10 includes a proximal flange 24 configured for engagement within a radially enlarged recessed section 26 of the axial bore 18 of the body portion 16 for easy assembly.

The body portion 16 is formed from a flexible material, such as silicone or another similar bio-compatible elastomeric material, and the axial bore 18 of the body portion 16 has a radially inwardly tapered section 28 that aids in diametrical expansion of the axial bore 18, as best seen in FIG. 8. In addition, the body portion 16 has internal strain relief features 15 best seen in FIGS. 6 and 7, to accommodate the radial expansion of the axial bore 18.

As best seen in FIG. 1, the proximal housing 12 of vascular introducer 10 preferably has an end cap 34 that supports a hemostatic seal 36 for fluidly sealing the axial passageway 14 that communicates with the central lumen 22 of the tubular sheath 20. In addition, the proximal housing 12 of vascular introducer 10 includes a radially extending hub 35 for accommodating a 3-way stopcock valve, which is not shown.

It is envisioned that the proximal housing 12 and the end cap 34 can be splittable into two halves along a diametrical parting line 38 to aide in removing the tubular sheath 20 from the operative site without having to remove an instrument or device extending therethrough. In such an instance, the tubular sheath 20 would also be splittable along its length.

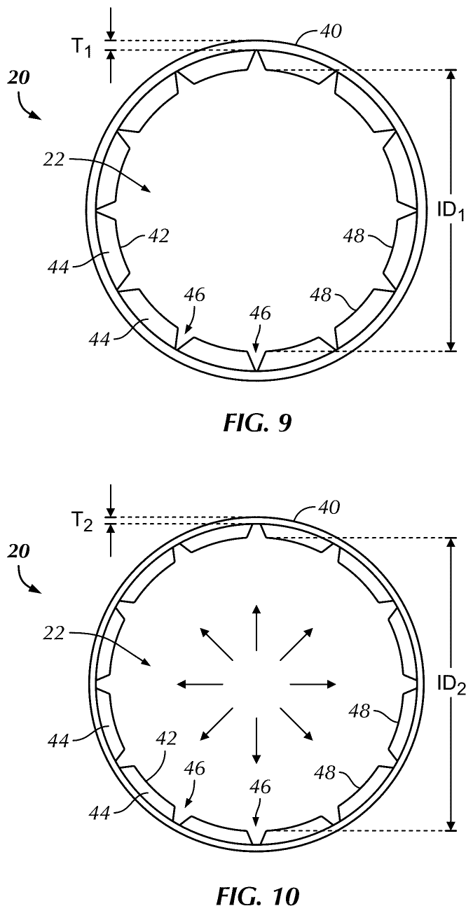

Referring now to FIGS. 9 and 10, the tubular sheath 20 of vascular introducer 10 includes an outer sheath portion 40 and an inner sheath portion 42. The outer sheath portion 40 of the tubular sheath 20 is defined by an elongated flexible tube extruded from a soft pliable material. In an embodiment of the invention, the outer sheath portion 40 has an initial thickness T₁ of about 0.006 inches. The inner sheath portion 42 of the tubular sheath 20 is defined by a scored extrusion having a plurality of circumferentially spaced apart elongated struts 44.

The scored extrusion that defines the inner sheath portion 42 of the tubular sheath 20 is preferably formed from high-density polyethylene (HDPE) or a similar hard plastic material such as a polyamide or similar nylon material. As shown, in some examples, the inner sheath portion 42 includes twelve (12) circumferentially spaced-apart elongated rigid struts 44, each separated by a longitudinally extending triangular score line or groove 46 and each one having an arcuate inner surface 48.

Those skilled in the art will readily appreciate that the number of rigid struts formed in the inner sheath portion 42 of the tubular sheath can be less than 12 or greater than 12, depending upon the dimensions and design of the sheath 20. For example, the inner sheath portion, in some examples, can have four (4) circumferentially spaced apart elongated rigid struts 44 to promote an even radial expansion of the outer sheath portion 40.

Preferably, the axial bore 18 of the body portion 16 and the central lumen 22 of the tubular sheath 20 is adapted and configured to transition between a first inner diameter ID₁ of 14 F and a second inner diameter ID₂ of 18 F. During such a transition, the outer sheath portion 40 is reduced from the initial thickness T₁ to a subsequent thickness T₂ of about 0.003 inches.

Those skilled in the art will readily appreciate that the first inner diameter ID₁ of the tubular sheath 20 of vascular introducer can be greater or less than 14 F, and that the second inner diameter ID₂ of the tubular sheath 20 of vascular introducer 10 can be greater or less than 18 F, without departing from the spirit or scope of the subject disclosure. Moreover, it should be appreciated that sheath sizes for vascular introducers can vary from 12 F or less to 24 F or greater.

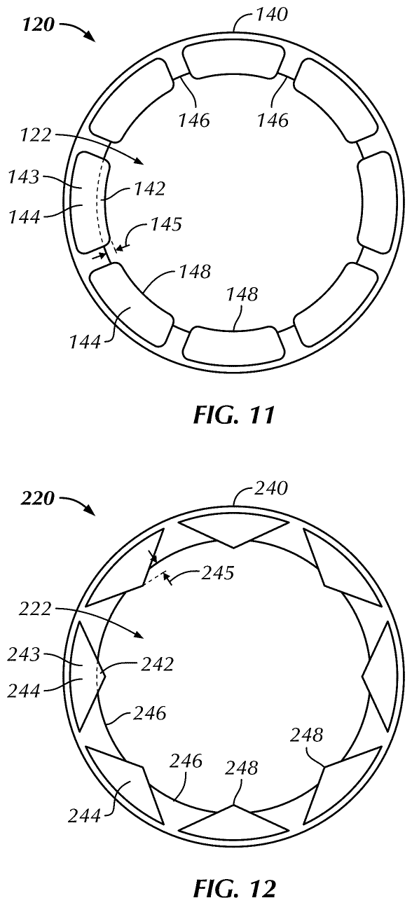

Referring now to FIGS. 11-15, in various examples, different configurations of struts or ribs 144, 244, 344, 444, 544 are contemplated for use with a vascular introducer similar to the vascular introducer 10 described herein and shown in FIGS. 1 and 2. Although each example is described and shown herein having multiple similarly-configured struts or ribs 144, 244, 344, 444, 544 (that is, similar shape, size, etc.), it should be understood that, in various examples, two or more configurations of struts or ribs 144, 244, 344, 444, 544 can be used in the same vascular introducer, depending upon the desired characteristics for the particular vascular introducer. It is also noted that although eight evenly-spaced struts or ribs 144, 244, 344, 444, 544 are shown in each of the examples of FIGS. 11-15, this is not intended to be limiting. As such, in other examples, it is contemplated that the vascular introducer includes more or fewer than eight struts or ribs 144, 244, 344, 444, 544, and/or different spacing between adjacent struts or ribs 144, 244, 344, 444, 544, depending upon the desired mechanical characteristics of the vascular introducer.

Referring still to FIGS. 11-15, in some examples, a vascular introducer similar to the vascular introducer 10 described and shown herein includes an elongated tubular sheath 120, 220, 320, 420, 520 extending distally from a proximal housing similar to the proximal housing 12 described and shown herein. In some examples, the tubular sheath 120, 220, 320, 420, 520 includes a sheath wall 140, 240, 340, 440, 540 defining a central lumen 122, 222, 322, 422, 522. In some examples, the central lumen 122, 222, 322, 422, 522 of the tubular sheath 120, 220, 320, 420, 520 includes an unexpanded configuration (for instance, similar to the tubular sheath 20 with the first inner diameter ID₁ described above and shown in FIG. 9) and an expanded configuration (for instance, similar to the tubular sheath 20 with the second inner diameter ID₂ described above and shown in FIG. 10). In some examples, the central lumen 122, 222, 322, 422, 522 of the tubular sheath 120, 220, 320, 420, 520 in the unexpanded configuration includes a first inner diameter (similar to the first inner diameter ID₁ described above and shown in FIG. 9), and the central lumen 122, 222, 322, 422, 522 of the tubular sheath 120, 220, 320, 420, 520 in the expanded configuration includes a second inner diameter (similar to the second inner diameter ID₂ described above and shown in FIG. 10), the second inner diameter being larger than the first inner diameter. In some examples, the sheath wall 140, 240, 340, 440, 540 is formed from an expandable polymeric material (in some examples, chronoprene, for instance) to allow expansion of the central lumen 122, 222, 322, 422, 522 of the tubular sheath 120, 220, 320, 420, 520 from the unexpanded configuration to the expanded configuration and/or allow contraction from the expanded configuration back down to the unexpanded configuration. In some examples, the central lumen 122, 222, 322, 422, 522 of the tubular sheath 120, 220, 320, 420, 520 is configured to transition between the first inner diameter and the second inner diameter in response to introduction of a device or object (such as, for instance, the dilator 30 described herein and shown in FIGS. 2 and 3) within the central lumen 122, 222, 322, 422, 522. In some examples, the device includes an outer diameter that is greater than the first inner diameter of the central lumen 122, 222, 322, 422, 522 of the tubular sheath 120, 220, 320, 420, 520.

In some examples, the tubular sheath 120, 220, 320, 420, 520 includes a plurality of circumferentially spaced apart elongated ribs or struts 144, 244, 344, 444, 544 disposed partially within the sheath wall 140, 240, 340, 440, 540, such that an exposed portion 142, 242, 342, 442, 542 of each of the plurality of struts 144, 244, 344, 444, 544 extends radially inwardly a distance 145, 245, 345, 445, 545 from an inner surface of the sheath wall 140, 240, 340, 440, 540 into the central lumen 122, 222, 322, 422, 522. The ribs or struts 144, 244, 344, 444, 544, in some examples, are formed from one or more stiff, non-expandable materials. In some examples, the tubular sheath 120, 220, 320, 420, 520 includes the ribs or struts 144, 244, 344, 444, 544 running substantially straight along a length of the tubular sheath 120, 220, 320, 420, 520. The ribs or struts 144, 244, 344, 444, 544, in some examples, run substantially the entire length of the tubular sheath 120, 220, 320, 420, 520. In other examples, the ribs or struts 144, 244, 344, 444, 544 run along a portion or portions of the length of the tubular sheath 120, 220, 320, 420, 520. In some examples, the ribs or struts 144, 244, 344, 444, 544 are disposed within the tubular sheath 120, 220, 320, 420, 520 substantially parallel to one another. In some examples, the struts 144, 244, 344, 444, 544 are substantially uniformly spaced within the tubular sheath 120, 220, 320, 420, 520 in a circular pattern around the tubular sheath 120, 220, 320, 420, 520. In some examples, the number of struts 144, 244, 344, 444, 544 in various tubular sheaths 120, 220, 320, 420, 520 can be variable, such as, for instance one or more struts 144, 244, 344, 444, 544 disposed within the tubular sheath 120, 220, 320, 420, 520. In further examples, various tubular sheaths 120, 220, 320, 420, 520 can include two, three, four, six, eight, ten, or twelve struts 144, 244, 344, 444, 544 disposed within the tubular sheath 120, 220, 320, 420, 520. In some examples, varying the size, number, and/or shape of the struts 144, 244, 344, 444, 544 can increase or decrease column strength and/or resistance to torsional forces of the tubular sheath 120, 220, 320, 420, 520. For instance, fewer struts 144, 244, 344, 444, 544 and/or larger struts 144, 244, 344, 444, 544 can lead to increased stiffness and column strength of the tubular sheath 120, 220, 320, 420, 520. Additionally, manipulating the width of each strut 144, 244, 344, 444, 544 and the width of the gaps between the struts 144, 244, 344, 444, 544 can increase or decrease the expansion force and column strength of the tubular sheath 120, 220, 320, 420, 520. For instance, increasing the width of the struts 144, 244, 344, 444, 544 decreases the width of the gaps between the struts 144, 244, 344, 444, 544 and increases both expansion force and column strength of the tubular sheath 120, 220, 320, 420, 520. In turn, decreasing the width of the struts 144, 244, 344, 444, 544 increases the width of the gaps between the struts 144, 244, 344, 444, 544 and decreases both expansion force and column strength of the tubular sheath 120, 220, 320, 420, 520.

In some examples, varying the amount of the expandable polymeric material of the tubular sheath 120, 220, 320, 420, 520 between the struts 144, 244, 344, 444, 544 can increase (smaller gap, less expandable material) or decrease (larger gap, more expandable material) an expansion force required to expand the tubular sheath 120, 220, 320, 420, 520 as the device or object passes through the central lumen 122, 222, 322, 422, 522. Additionally, in some examples, varying a thickness of the sheath wall 140, 240, 340, 440, 540 can increase or decrease the force required to expand the sheath. That is, in some examples, a thicker sheath wall 140, 240, 340, 440, 540 results in a higher force required to expand the tubular sheath 120, 220, 320, 420, 520 while a thinner sheath wall 140, 240, 340, 440, 540 results in a lower force required to expand the tubular sheath 120, 220, 320, 420, 520.

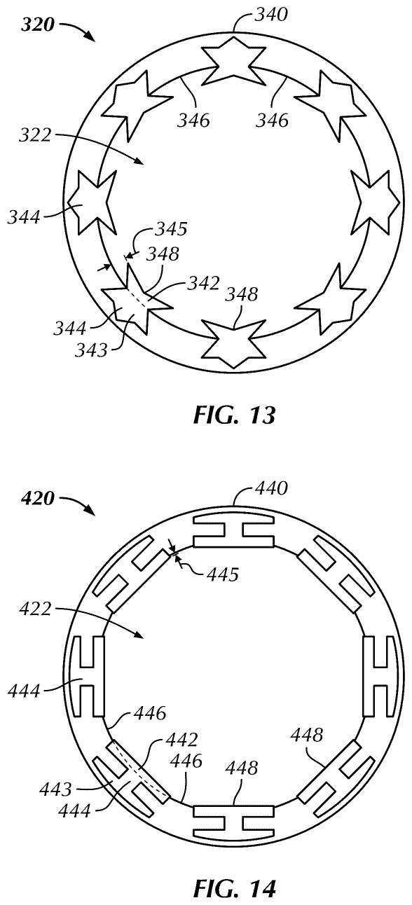

In various examples, the distance 145, 245, 345, 445, 545 that each of the struts 144, 244, 344, 444, 544 extends from the inner surface of the sheath wall 140, 240, 340, 440, 540 can vary, as can be seen in FIG. 13 with the relatively large distance 345 that the struts 344 extend inwardly from the inner surface of the sheath wall 340 as compared to the relatively small distance 445 that the struts extend inwardly from the inner surface of the sheath wall 440 in FIG. 14, depending upon the application, the desired size, and/or, the desired characteristics of the vascular introducer. Each of the struts 144, 244, 344, 444, 544, in some examples, includes an embedded portion 143, 243, 343, 443, 543 disposed within the sheath wall 140, 240, 340, 440, 540. The embedded portion 143, 243, 343, 443, 543, in some examples, provides a mechanical connection between the struts 144, 244, 344, 444, 544 and the sheath wall 140, 240, 340, 440, 540. In addition to or instead of being embedded within the sheath wall 140, 240, 340, 440, 540, the struts 144, 244, 344, 444, 544 can be coupled to the sheath wall 140, 240, 340, 440, 540 using, an adhesive, one or more fasteners, a combination thereof, or the like.

In some examples, the exposed portion 142, 242, 342, 442, 542 of each of the plurality of struts 144, 244, 344, 444, 544 provides a contact area 148, 248, 348, 448, 548 along which the device (such as, for instance, the dilator 30 described herein and shown in FIGS. 2 and 3) inserted within the central lumen 122, 222, 322, 422, 522 can slide. In some examples, the total amount of contact area 148, 248, 348, 448, 548 across all of the struts 144, 244, 344, 444, 544 is less than a surface area of an interior of the tubular sheath 120, 220, 320, 420, 520 to decrease frictional resistance experienced by the device inserted within the central lumen 122, 222, 322, 422, 522. In some examples, the contact area 148, 248, 348, 448, 548 of each of the plurality of struts 144, 244, 344, 444, 544 comes into contact with the objects and/or devices as they pass through the tubular sheath 120, 220, 320, 420, 520 and expand the central lumen 122, 222, 322, 422, 522. In some examples, by creating smaller contact areas 148, 248, 348, 448, 548 between the central lumen 122, 222, 322, 422, 522 and the object or device passing though the tubular sheath 120, 220, 320, 420, 520, the force required to pass the object or device through the tubular sheath 120, 220, 320, 420, 520 is reduced. In some examples, at least the contact area 148, 248, 348, 448, 548 of each of the plurality of struts 144, 244, 344, 444, 544 is formed from a lubricious material to further decrease frictional resistance experienced by the device when inserted within the central lumen 122, 222, 322, 422, 522. In some examples, the struts 144, 244, 344, 444, 544 extending from the inner surface of the sheath wall 140, 240, 340, 440, 540 by the distance 145, 245, 345, 445, 545 lessens, if not eliminates, the chances of the device inserted within the central lumen 122, 222, 322, 422, 522 from contacting the sheath wall 140, 240, 340, 440, 540. The sheath wall 140, 240, 340, 440, 540, being formed from the expandable polymeric material, would increase frictional resistance if the device inserted within the central lumen 122, 222, 322, 422, 522 were to slide along the sheath wall 140, 240, 340, 440, 540, given the higher frictional resistance of an expandable polymeric material as compared to that of a lubricious material. By providing the struts 144, 244, 344, 444, 544 separated from the inner surface of the sheath wall 140, 240, 340, 440, 540 by the distance 145, 245, 345, 445, 545, in some examples, it allows the device inserted within the central lumen 122, 222, 322, 422, 522 to slide along only the contact areas 148, 248, 348, 448, 548 and avoid contact with the sheath wall 140, 240, 340, 440, 540, thereby decreasing the total surface area along which the device slides (comprising the surface area of the contact areas 148, 248, 348, 448, 548 only since there is empty space between the contact areas 148, 248, 348, 448, 548) and, in some examples, due to the lubricious material forming at least the contact areas 148, 248, 348, 448, 548 of the struts 144, 244, 344, 444, 544 and the decreased surface area along which the device slides, decreasing the frictional resistance experienced by the device when inserted into and slid within the central lumen 122, 222, 322, 422, 522 of the tubular sheath 120, 220, 320, 420, 520. That is, in some examples, with the contact area 148, 248, 348, 448, 548 of the struts 144, 244, 344, 444, 544 protruding farther into central lumen 122, 222, 322, 422, 522 than the expandable polymeric material of the sheath wall 140, 240, 340, 440, 540 and with the material of the struts 144, 244, 344, 444, 544 being generally more lubricious than the expandable polymeric material of the sheath wall 140, 240, 340, 440, 540, the objects and devices passing through the central lumen 122, 222, 322, 422, 522 of the tubular sheath 120, 220, 320, 420, 520 contact only the lubricious material of the contact area 148, 248, 348, 448, 548 of the struts 144, 244, 344, 444, 544, thereby reducing friction and, in turn, the force required to pass the objects and devices through the tubular sheath 120, 220, 320, 420, 520.

Referring now to FIG. 11, in some examples, a cross section of at least one of the plurality of struts 144 of the tubular sheath 120 includes a substantially rectangular shape. In some examples, the substantially rectangular shape of the one or more struts 144 need not be perfectly rectangular and, instead, can include rounded corners and/or slightly curved sides. In some examples, the substantially rectangular shape of the one or more struts 144 can include other quadrilaterally-shaped cross sections, such as rhombus shaped, square shaped, parallelogram shaped, or trapezoid shaped, for instance. In some examples, the number and/or width of the struts 144 can be altered to allow for bigger gaps in between struts 144 (smaller and/or fewer struts 144 in the tubular sheath 120) to decrease expansion force and column strength of the tubular sheath 120 or smaller gaps in between struts 144 (larger and/or more struts 144 in the tubular sheath 120) to increase expansion force and column strength of the tubular sheath 120. In some examples, smaller and/or fewer struts 144 in the tubular sheath 120 provides a smaller total surface area of the contact areas 148, thereby decreasing frictional resistance experienced by objects and/or devices inserted within the central lumen 122 of the tubular sheath 120 and, in turn, decreasing the force necessary to insert and slide the object or device within the central lumen 122 of the tubular sheath 120. In other examples, larger and/or more struts 144 in the tubular sheath 120 provides a larger total surface area of the contact areas 148, thereby increasing frictional resistance experienced by objects and/or devices inserted within the central lumen 122 of the tubular sheath 120 and, in turn, increasing the force necessary to insert and slide the object or device within the central lumen 122 of the tubular sheath 120.

Referring now to FIG. 12, in some examples, a cross section of at least one of the plurality of struts 244 of the tubular sheath 220 includes a substantially triangular shape. In some examples, this triangular configuration allows for more expandable polymeric material on an inner portion of the sheath wall 240 of the tubular sheath 220 and more material of the struts 244 toward an outer portion of the sheath wall 240 of the tubular sheath 220, resulting in less force required to expand the tubular sheath 220 as an object or device passes through the central lumen 222 while maximizing column strength of the tubular sheath 220 by allowing for more strut material toward the outer portion of the sheath wall 240. In some examples, the triangular shape of the struts 244 allows for the contact area 248 of each of the struts 244 to be a point of each of the triangular struts 244. In this way, the total surface area of the contact areas 248 of the struts 244 along which objects and/or devices slide when inserted within the central lumen 222 of the tubular sheath 220 is substantially minimized, thereby greatly reducing frictional resistance experienced by objects and/or devices inserted within the central lumen 222 of the tubular sheath 220 and, in turn, decreasing the force necessary to insert and slide the object or device within the central lumen 222 of the tubular sheath 220.

Referring now to FIG. 13, in some examples, a cross section of at least one of the plurality of struts 344 of the tubular sheath 320 is substantially star-shaped. In some examples, the star-shaped struts 344 allow for maximizing the amount of expandable polymeric material of the sheath wall 340 in between the struts 344 while still retaining higher column strength of the tubular sheath 320 due to the relatively large cross-sectional area of the struts 344.

Referring now to FIG. 14, in some examples, a cross section of at least one of the plurality of struts 444 of the tubular sheath 420 is substantially I-shaped. In some examples, I-shaped struts 444 allow for an alternative configuration with higher column and torsional strength than would be possible with a differently-shaped struts, such as, for instance, the rectangular-shaped struts 144 described herein.

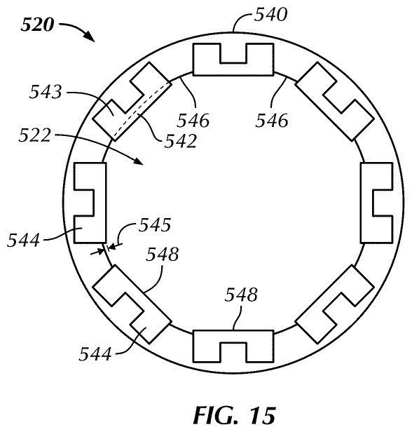

Referring now to FIG. 15, in some examples, a cross section of at least one of the plurality of struts 544 of the tubular sheath 520 is substantially U-shaped. In some examples, U-shaped struts 544 allow for an alternative configuration with higher column and torsional strength than would be possible with a differently-shaped struts, such as, for instance, the rectangular-shaped struts 144 described herein.

It should be understood that, in other examples, struts having shapes other than those described and shown herein are contemplated, such as, but not limited to struts having a circular cross section, an elliptical cross section, a polygonal cross section having five sides or greater, a dog bone cross section, or the like.

It is envisioned that the vascular introducer 10 of the subject invention may be provided for use and/or sale as a kit that would include an enclosure or sealed tray containing a dynamically expandable vascular introducer 10 as described above, and at least one dilator 30 having a shaft with an outer diameter OD ranging from about 14 F to about 18 F. Preferably, the kit would include at least one dilator 30 having a shaft 32 with an outer diameter OD of about 14 F and a second dilator 30 having a shaft 32 with an OD that is greater than 14 F, for example, it could have a shaft 32 with an OD of 18 F. While the subject disclosure has been shown and described with reference to preferred embodiments, those skilled in the art will readily appreciate that changes or modifications may be made thereto without departing from the spirit or scope of the subject disclosure.

The present inventors have recognized various advantages of the subject matter described herein. The present inventors have recognized, among other things, that the present subject matter can be used to provide a vascular introducer with the ability to expand and contract in response to the insertion of different diameter therapeutic devices, thereby avoiding the need to remove the initial access introducer from the surgical access point in order to introduce larger sheaths/introducers thereafter. In various examples, the present inventive subject matter is advantageous in that it provides a vascular introducer including a lubricious material in the device lumen or inner diameter (ID) and/or various rib shapes and configurations to allow for easier passage of dilators and devices, for instance, due to decreased frictional resistance and insertion force during passage of a device within the vascular introducer. In some examples, the present inventive subject matter provides for a vascular introducer including one or more various rib shape designs to allow for improved strength and performance within a limited size/wall thickness. While various advantages of the example systems are listed herein, this list is not considered to be complete, as further advantages may become apparent from the description and figures presented herein.

Although the subject matter of the present patent application has been described with reference to various examples, workers skilled in the art will recognize that changes can be made in form and detail without departing from the scope of the subject matter recited in the below claims.

The above Detailed Description includes references to the accompanying drawings, which form a part of the Detailed Description. The drawings show, by way of illustration, specific examples in which the present apparatuses and methods can be practiced. These embodiments are also referred to herein as “examples.”

The above Detailed Description is intended to be illustrative, and not restrictive. For example, the above-described examples (or one or more elements thereof) can be used in combination with each other. Other embodiments can be used, such as by one of ordinary skill in the art upon reviewing the above description. Also, various features or elements can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

In this document, the terms “a” or “an” are used to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “about” and “approximately” or similar are used to refer to an amount that is nearly, almost, or in the vicinity of being equal to a stated amount.

In the appended claims, the terms “including” and “in which” are used as the plain-English equivalents of the respective terms “comprising” and “wherein.” Also, in the following claims, the terms “including” and “comprising” are open-ended, that is, an apparatus or method that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

1. A vascular introducer comprising:
a proximal housing defining an axial passageway; and
an elongated tubular sheath extending distally from the proximal housing and including a sheath wall defining a central lumen, wherein the central lumen of the tubular sheath includes an unexpanded configuration and an expanded configuration, wherein the central lumen of the tubular sheath in the unexpanded configuration includes a first inner diameter and the central lumen of the tubular sheath in the expanded configuration includes a second inner diameter, the second inner diameter being larger than the first inner diameter, the tubular sheath including a plurality of circumferentially spaced apart elongated struts disposed partially within the sheath wall, such that an exposed portion of each of the plurality of struts extends radially inwardly from an inner surface of the sheath wall into the central lumen.

2. The vascular introducer of claim 1, wherein a cross section of at least one of the plurality of struts includes a substantially rectangular shape.

3. The vascular introducer of claim 1, wherein a cross section of at least one of the plurality of struts includes a substantially triangular shape.

4. The vascular introducer of claim 1, wherein a cross section of at least one of the plurality of struts is substantially star-shaped.

5. The vascular introducer of claim 1, wherein a cross section of at least one of the plurality of struts is substantially U-shaped.

6. The vascular introducer of claim 1, wherein a cross section of at least one of the plurality of struts is substantially I-shaped.

7. The vascular introducer of claim 1, wherein the central lumen of the tubular sheath is configured to transition between the first inner diameter and the second inner diameter in response to introduction of a device within the central lumen, the device including an outer diameter that is greater than the first inner diameter of the central lumen of the tubular sheath.

8. The vascular introducer of claim 1, comprising a body portion extending distally from the proximal housing and defining an axial bore in communication with the axial passageway of the proximal housing, wherein the tubular sheath extends distally from the axial bore of the body portion.

9. The vascular introducer of claim 1, wherein the exposed portion of each of the plurality of struts provides a contact area along which a device inserted within the central lumen slides, the contact area being less than a surface area of an interior of the tubular sheath to decrease frictional resistance experienced by the device inserted within the central lumen.

10. The vascular introducer of claim 9, wherein at least the contact area of each of the plurality of struts is formed from a lubricious material to decrease frictional resistance experienced by the device inserted within the central lumen.

11. The vascular introducer of claim 1, wherein the body portion is formed from a flexible material.

12. The vascular introducer of claim 1, wherein the sheath wall is formed from an expandable polymeric material.

13. A vascular introducer comprising:
a proximal housing defining an axial passageway; and
an elongated tubular sheath extending distally from the proximal housing and including a sheath wall defining a central lumen, the sheath wall being formed from an expandable polymeric material, wherein the central lumen of the tubular sheath includes an unexpanded configuration and an expanded configuration, wherein the central lumen of the tubular sheath in the unexpanded configuration includes a first inner diameter and the central lumen of the tubular sheath in the expanded configuration includes a second inner diameter, the second inner diameter being larger than the first inner diameter, the tubular sheath including a plurality of circumferentially spaced apart elongated struts disposed partially within the sheath wall, such that an exposed portion of each of the plurality of struts extends radially inwardly from an inner surface of the sheath wall into the central lumen, the exposed portion of each of the plurality of struts providing a contact area along which a device inserted within the central lumen slides, the contact area being less than a surface area of an interior of the tubular sheath, at least the contact area of each of the plurality of struts being formed from a lubricious material, such that frictional resistance experienced by the device inserted within the central lumen is decreased.

14. The vascular introducer of claim 13, wherein a cross section of at least one of the plurality of struts includes a substantially rectangular shape.

15. The vascular introducer of claim 13, wherein a cross section of at least one of the plurality of struts includes a substantially triangular shape.

16. The vascular introducer of claim 13, wherein a cross section of at least one of the plurality of struts is substantially star-shaped.

17. The vascular introducer of claim 13, wherein a cross section of at least one of the plurality of struts is substantially U-shaped.

18. The vascular introducer of claim 13, wherein a cross section of at least one of the plurality of struts is substantially I-shaped.

19. The vascular introducer of claim 13, wherein the central lumen of the tubular sheath is configured to transition between the first inner diameter and the second inner diameter in response to introduction of a device within the central lumen, the device including an outer diameter that is greater than the first inner diameter of the central lumen of the tubular sheath.

20. A vascular introducer comprising:
a proximal housing defining an axial passageway;
a body portion extending distally from the proximal housing and defining an axial bore in communication with the axial passageway of the proximal housing, the body portion being formed from a flexible material; and
an elongated tubular sheath extending distally from the axial bore of the body portion and including a sheath wall defining a central lumen, the sheath wall being formed from an expandable polymeric material, wherein the central lumen of the tubular sheath includes an unexpanded configuration and an expanded configuration, wherein the central lumen of the tubular sheath in the unexpanded configuration includes a first inner diameter and the central lumen of the tubular sheath in the expanded configuration includes a second inner diameter, the second inner diameter being larger than the first inner diameter, the tubular sheath including a plurality of circumferentially spaced apart elongated struts disposed partially within the sheath wall, such that an exposed portion of each of the plurality of struts extends radially inwardly from an inner surface of the sheath wall into the central lumen, the exposed portion of each of the plurality of struts providing a contact area along which a device inserted within the central lumen slides, the contact area being less than a surface area of an interior of the tubular sheath, at least the contact area of each of the plurality of struts being formed from a lubricious material, such that frictional resistance experienced by the device inserted within the central lumen is decreased.
